(19) United States
(12) Patent Application Publication
(10) Pub. No.: US 2026/0053657 A1
(43) Pub. Date: Feb. 26, 2026

(54) FIXATION MEMBER FOR AFFIXATION TO A STRUCTURE IN AN ORTHOPEDIC DEVICE AND METHOD FOR SECURING THE SAME

(71) Applicant: Ossur Iceland ehf, Reykjavik (IS)

(72) Inventors: Sigmar Darri UNNSTEINSSON, Reykjavik (IS); David SANDAHL, Reykjavik (IS)

(21) Appl. No.: 19/306,119

(22) Filed: Aug. 21, 2025

Related U.S. Application Data
(60) Provisional application No. 63/685,904, filed on Aug. 22, 2024.

(51) Int. Cl. A61F 5/055

(52) U.S. Cl. CPC A61F 5/055

(57) ABSTRACT

A fixation member, an orthopedic device using a fixation member, or a method for using the same includes fastener elements that are integrally formed with the fixation member that can be attached to the orthopedic device without adhesive. The fixation member may be secured to the orthopedic device through welding and/or a mechanical connection. The welding attachment may involve a weld created through various processes, including a heat staking process.

### CROSS REFERENCE TO RELATED APPLICATIONS

This disclosure incorporates by reference U.S. provisional application no. 63/685,904, filed on August 22, 2024; U.S. patent no. 9,474,334, granted on October 25, 2016; U.S. patent no. 10,245,170, granted on April 2, 2019; and U.S. patent no. 10, 945872, granted on March 16, 2021.

### FIELD OF THE DISCLOSURE

The disclosure relates to a fixation member adapted for a structure, and more specifically, to a fixation member having molded hook elements arranged for permanently attaching to an orthopedic device or suitable article for receiving corresponding loop material.

### BACKGROUND

Hook and loop fasteners are a two-component fastening system that comprises a hook component and a loop component configured to interlock together to form a connection. Hook and loop fasteners are commonly chosen because they form strong attachments, are easily and quickly adjustable, and, in cases where the hook and loop fasteners are formed on strap material, are easily incorporated into many articles.

Hook and loop fasteners often comprise two strap portions – a hook strap portion with a plurality of hook elements and a loop strap portion with a plurality of loop elements, which are arranged to catch and interlock with the plurality of loop elements. The hook and loop elements can be unfastened by pulling on one of the strap portions. In this manner, the hook strap portion is releasably secured to the loop strap portion. The hook elements may be formed from plastic, while the loop elements may be formed from a fabric or other fibrous material, such as a plastic filament or cotton or wool fibers.

The hook and loop fastener may be used to secure two structures together. The hook and loop strap portions may form separate portions of a single strap. In these instances, a first strap portion of the hook and loop strap portions (often the hook strap portion) is attached to a first structure. A second strap portion (i.e., the other of the hook and loop strap portions – often the loop strap portion) is threaded through a strap loop attached to a second structure. The second strap portion is then attached to the first strap portion to secure the first structure to the second structure releasably. In other instances, the hook strap portion may be attached to the first structure, and the loop strap portion may be attached to the second structure in a location arranged to interface with the hook strap portion, to secure the first and second structures together. Hook and loop fasteners are often applied in wearable articles (e.g., shoes, backpacks, pocket flaps, protective sports equipment, etc.), organizational management (e.g., to secure tools and handheld equipment), medical supplies, etc.

It is common practice to use adhesives to attach the hook and loop strap portions to the underlying structures. It is often found, however, that the shear forces applied to the adhesives (e.g., from the fastening-unfastening cycles in using the hook and loop fastener) and temperature and humidity fluctuations can cause the strap portions to slide and migrate throughout use. This potential occurrence may cause problems, such as the underlying structures being improperly secured to one another or the hook and/or loop strap portions becoming detached from the underlying structures, thereby preventing attachment of the underlying structures. In some cases, the hook or loop strap portions are incorporated into an article by a sewn connection rather than by an adhesive, wherein the strap portion is sewn via thread directly to the article. However, many articles comprise materials (e.g., metals or plastics) that are too thick or hard for sewn attachments.

In the past, injection-molded hooks integrally molded onto the underlying structures were used to prevent the hook elements from sliding and coming loose from the underlying structure. This practice involves placing an insert for the hook elements into an injection molding tool for the underlying structure. An integrally formed field of hooks is then created during the molding or manufacturing process, molding the hook directly into the plastic of the underlying structure. This method eliminates the need for adhesively applying the hook mentioned above to material pieces and reduces the step of applying hook material pieces later in fabricating an article. However, it may not be feasible to provide injection molded hooks on some articles, as some articles may require molding of the underlying structure, such that the molded device comprises contoured surfaces to which the insert may not conform.

Hook and loop fasteners are commonly incorporated into orthopedic devices to secure the device to a user. The flexibility of the hook and loop strap portions enables a user to quickly fasten and unfasten the orthopedic device to a portion of the user's body, enabling quick device adjustment. However, known attachment mechanisms used to connect hook and loop fasteners may not be feasible with orthopedic devices. Orthopedic devices often comprise hard, rigid materials that either do not allow for sewn connections or wherein sewn connections are insufficient to secure the device to the user properly.

While injection molding the hook elements directly onto many articles is effective, it suffers from the drawback that many orthopedic devices do not include integrally molded hook elements, particularly for molded orthopedic devices. For example, knee brace shells are commonly post-formed onto a user's leg mold after the molded hooks of injection molded inserts are formed by injection molding, rendered in a flat configuration. Because the underlying structure of the orthopedic device may be subsequently contoured to the user after the insert itself is formed, integrally molded hook elements may become damaged during the post-forming and customization process. Due to the inherent differences in the shape of each user, the integrally molded hook elements may be at a less favorable location after the underlying structure has been formed. Known methods for integrally molding hook elements lack flexibility for later modification in location and limit the degree to which the underlying structure may be customized for an individual user.

Some orthopedic devices employ hook and loop fasteners attached to the device by adhesives. However, similarly as described above, the hook and loop fasteners may delaminate from or migrate along the orthopedic device as the adhesive deteriorates from, for example, shear forces applied to the adhesive. This may lead to an improper fit of the orthopedic device on the user's body and reduce the device's effectiveness. The adhesive may also deteriorate from temperature and humidity fluctuations, which may be particularly exacerbated by the proximity of the hook and loop fastener to portions of the body (e.g., from sweat or heat imparted to the fastener from the user's body). Adhesives pose additional problems in orthopedic devices. Many adhesives emit strong or offensive odors, which may decrease the user's comfort and compliance with the device. Also, adhesives may reduce the control the end manufacturer has over the construction of the device. Because many devices are assembled in whole or in part in different parts of the world, changes to the type of adhesive used may not be communicated to or noticed within sufficient time by the end manufacturer. This development may lead to decreased user satisfaction if the adhesive is not configured to the device's requirements. Thus, there exists a need for new designs and methods for securing hook and loop fasteners to articles without the need for adhesives.

### SUMMARY

Disclosed are embodiments of a fixation member comprising molded fastening elements and a method of attaching the same to an article's underlying structure (i.e., "structure"). Importantly, the fixation member may be attached to the structure without using fasteners that do not form a part of the fixation member or underlying structure. Specifically, the fixation member may be attached to the structure without the use of adhesive, as well as other fasteners. The fixation member may be affixed to the structure by a mechanical connection and/or a welded attachment.

The fixation member may comprise a plastic material formed from a material used in injection-molded processes, such as a thermoplastic or thermoset material. The fixation member may comprise a base including first and second sides and first and second ends. The base may resemble a plate and may be flat or may have a contoured profile. The base can be formed of a rigid or a flexible material, or may have flexibility and stiffness adapted according to the requirements of the underlying structure or the needs of the user. The base may be formed so as not to interfere substantially with the underlying structure to which the fixation member is attached.

A plurality of fastening elements may be integrally formed with and extend from the first side of the base. The plurality of fastening elements may be formed from an injection-molded process. According to an embodiment, the plurality of fastening elements may comprise hook elements and/or loop elements integrally formed with the base of the fixation member. The fastening elements may comprise other integrally formed fasteners, such as clamps or other fasteners. The plurality of fastening elements may comprise multiple types of fastening elements. The plurality of fastening elements may protrude from a surface extending over a majority of the first side or from a surface extending over a minority of the first side. The portion of the first side over which the plurality of fastening elements does not protrude may comprise a smooth surface or a non-smooth surface, such as a rough surface or a surface shaped to conform to or interface with the underlying structure.

The second side of the base may comprise first and/or second fixation elements arranged to connect the fixation member with the structure. The first fixation elements may be arranged to form a welded attachment to an attachment feature of the structure through a welding process. The welding process may comprise a heat staking, ultrasonic welding, laser welding, spin or friction welding, solvent welding, extrusion welding, or hot plate welding, or other welding processes. The first fixation elements may be arranged in a first shape and configured such that upon exertion of heat, the fixation elements are formed into a second shape that interlocks with the attachment feature of the structure. The structure may have a higher glass transition temperature than that of the first fixation element, such that the exertion of heat may be sufficient to melt or deform the first fixation element but not the structure to which it is attached. Alternatively, the exertion of heat applied to the first fixation element may be sufficient to melt and deform the first fixation element and the attachment feature, such that the first fixation element and the attachment feature are integrally formed. According to an embodiment, the first fixation elements may comprise a heat stake that may be deformed under a heat staking process. The fixation member may comprise one first fixation element or may comprise two or more first fixation elements. The first fixation elements may be disposed closer to the second end of the base than the first end of the base.

The second fixation elements may be arranged to form a mechanical connection with a connection feature of the structure. The second fixation elements may comprise one or more of various mechanical connection elements configured to connect or interlock with a corresponding connection feature of a structure. The mechanical connection between the fixation member and the structure may comprise a releasable mechanical connection. The second fixation elements may comprise a snap hook extending from the second side of the base, the snap hooks being configured to connect to a connection feature comprising a connection opening mechanically. The second fixation element may comprise a slot extending from the base at a first or second end, wherein the slot is configured to receive a connection feature comprising a protuberance of the structure. The second fixation may comprise a first aperture configured to interlock with a connection feature comprising a second aperture formed in the structure, the first and second apertures being arranged to receive a pin.

The first and second fixation elements may enable the fixation member to attach to the structure without any fasteners formed independent of the fixation member and structure, including an adhesive. The welded attachment and mechanical connection of the fixation member may increase the durability of the connection between the fixation member and the structure. It may also prevent or mitigate the migration of the fixation member relative to the structure, especially over repeated fastening-unfastening cycles. Lack of an adhesive or other fasteners may enable the connection between the fixation member and the structure to remain free of ferromagnetic materials and enable the device to be attached to a user during magnetic resonance imaging (MRI), x-ray imaging, computed tomography (CT) scan, or other medical imaging technique without substantially interfering with the imaging technique or apparatus. Lack of an adhesive or other fasteners may prevent the introduction of odors or smells to the device and may simplify the assembly of the device.

The fixation member may be configured to affix to an underlying structure comprising an orthopedic device. The orthopedic device may comprise a cervical collar, an orthopedic walking boot, a back support and/or brace, foot and/or ankle support, a knee brace, or an elbow brace. The orthopedic device may include an anterior component and a posterior component configured to be connected or secured to the anterior component with the cooperation of the fixation member. The anterior component and the posterior component may comprise separate and distinct elements of the orthopedic device or may form different portions of the same element. The fixation member may be arranged to secure the anterior component to the posterior component through cooperation of fastening elements (e.g., hook and loop fasteners) as part of a fastening system.

The anterior component may comprise a first part and a second part. The second part may generally be disposed posteriorly of the first part. It may comprise a plastic that is less rigid than that of the first part, the second part being arranged to prevent the more rigid portions of the first part from rubbing up against and causing discomfort to the user. The first part may be connected to the second part. The fixation member may be connected to both the first and second parts. The fixation member may be welded to one of the first and second parts and may form a mechanical connection with the other part of the first and second parts. The fixation member may be welded to or mechanically attached to both the first and second parts. The fixation member may be connected to one but not the other of the first and second parts. These connections between the fixation member and the anterior and/or posterior components may improve the connection between the anterior and posterior components and/or may improve the connection between the fixation member and the orthopedic device. These and other features, aspects, and advantages of the present disclosure will become better understood regarding the following description, appended claims, and accompanying drawings.

### BRIEF DESCRIPTION OF THE DRAWINGS

The drawing figures are not necessarily drawn to scale but are intended to provide a better understanding of the components thereof, serving as exemplary illustrations rather than limiting in scope. The figures illustrate exemplary configurations of an orthopedic device and integrally formed fixation member incorporated therein, and in no way limit the structures or configurations according to the present disclosure.

FIG. 1 is a perspective view of an exemplary orthopedic device known in the art as having a strap system to secure anterior and posterior components to one another.

FIG. 2 is an exemplary perspective view of a prior art fastener element configuration.

FIG. 3 is a perspective view of an exemplary orthopedic device, including an anterior component of the device and a fixation member attached thereto, according to the present disclosure.

FIG. 4 is a front perspective view of an exemplary fixation member.

FIG. 5 is a back perspective view of the exemplary fixation member of FIG. 3.

FIG. 6 is a back perspective view of a second part of the anterior component configured to attach to the fixation member chemically.

FIG. 7 is a front perspective view of a fixation member mechanically connected to the first part of the anterior component.

FIGS. 8A-8B, 9A-9B, 10A-10B, and FIGS. 11A-11B illustrate before and after images of the heat staking process, with various configurations of the thermal tip of the forming tool.

### DEFINITIONS

As used herein, the term "hook and loop fastener" and "hook and loop fastening system" may refer to a fastener including two surfaces configured to be releasably attached. The first of the two surfaces may include a plurality of hook elements or micro-hook features. The second of the two surfaces may comprise a plurality of loops configured to be caught by or interlock with the plurality of hook elements or micro-hook features to secure the two surfaces together.

As used herein, the term "structure" or "underlying structure" may refer to an article or device or a portion of a device to which a fixation member may be attached. The structure may comprise multiple components configured to be secured to one another.

As used herein, the term "fixation member" may refer to an element arranged to attach to an underlying structure as part of a fastening system. The fixation member may comprise integrally formed fastening elements arranged to secure multiple components (e.g., multiple components of the structure) together.

As used herein, the term "base" may refer to a portion of the fixation member from which the fastening elements and the first and/or second fixation elements extend. The base may comprise a plate and may have a generally flat or contoured surface and may be rigid or flexible according to the requirements of the device or the needs of the user.

As used herein, the term "fastening element" may refer to a feature extending from a side of the base and configured to secure two or more structures together as part of a fastening system. The fastening elements may comprise hook elements and/or loop elements.

As used herein, the term "welding," "welded," and "weld" may refer to a feature or process wherein a component is melted and deformed from a first shape to a second shape upon the exertion of heat. The second shape may be configured to interlock with another feature of the structure or device to form a weld or welded attachment. The welded attachment may be formed by thermally deforming a feature with a first shape such that the feature exhibits a second shape. The welded attachment may be a permanent connection in that the attachment may not be undone or detached from the structure without destroying or deforming the weld. A welded attachment may also comprise two or more sub-components that are melted or fused to form an integrally formed component. The welded attachment may be formed through local heating and cooling of the associated elements.

As used herein, the term "heat stake" may refer to a protrusion of a first element configured to be welded to a second element via a heat staking process to form a welded attachment between the first and second elements.

As used herein, the term "mechanical connection" may refer to a connection that is achieved by the interaction of separate interlocking components and may refer to a releasable connection.

As used herein, the term "releasable connection," "releasably attached," and associated variants may refer to a connection between two structures that may be reversed or undone without destroying the components of the connection or substantially decreasing the ability of the connection components to reform the connection. Conversely, the term "unreleasable connection" or "permanent connection" may refer to a connection that may only be reversed or undone by destroying the components of the connection.

As used herein, the term "orthopedic device" may refer to any device or equipment configured to correct deformities of a user or to restore or replace the function of the human skeletal system. The term "orthopedic device" may additionally refer to any other structure to which the fixation member (described below) may be attached.

As used herein, the terms "anterior" and "posterior" (e.g., as used in the terms "anterior component" and "posterior component") may refer to directions relative to a user's body. For example, an anterior component may be a component of a device positioned anteriorly or towards the front of the user. In contrast, a "posterior component" may be a component of a device positioned posteriorly or towards the rear of a user. Similarly, the terms "inner" and "outer" may refer to directions of the orthopedic device relative to the user, with the term "inner" referring to portions or directions of the orthopedic device lying towards the user and the term "outer" referring to portions or directions of the orthopedic device lying away from the user or facing away from the user.

As used herein, the term "injection molded hooks" may refer to hook elements protruding from an underlying structure formed using a continuous fiber injection molding process, such that the hook elements are integrally formed with the underlying structure.

As used herein, the term "snap hooks" may refer to a flexible member having a hook feature that may allow the snap hook to enter but prevent its escape from an opening.

### DETAILED DESCRIPTION OF VARIOUS EMBODIMENTS

A better understanding of different embodiments of the disclosure may be had from the following description read with the accompanying drawings in which like reference characters refer to like elements. While the disclosure is susceptible to various modifications and alternative constructions, certain illustrative embodiments are in the drawings and are described below. It should be understood, however, that there is no intention to limit the disclosure to the embodiments disclosed; on the contrary, the intention covers all modifications, alternative constructions, combinations, and equivalents falling within the spirit and scope of the disclosure.

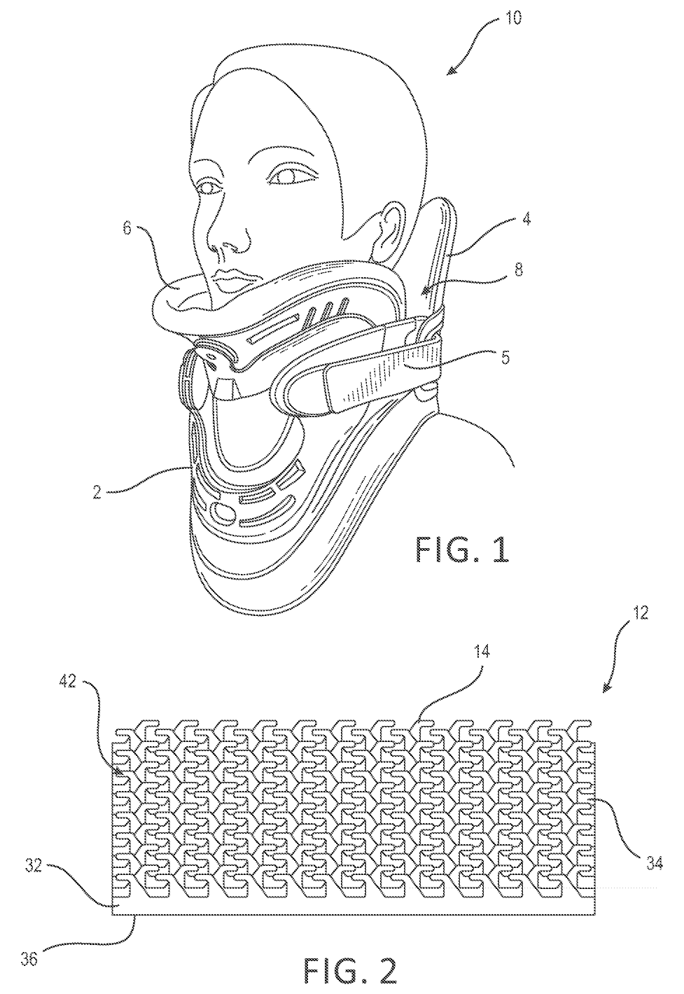

FIG. 1 shows an exemplary orthopedic device or article in which a hook and loop fastener 5 may be used. The illustrated orthopedic device comprises a cervical collar 10 with an anterior component 2 and a posterior component 4 configured to cooperate to support the head and neck of a user. Although the orthopedic device depicted comprises a cervical collar 10, hook and loop fasteners 5 have also been found useful in other orthopedic devices, such as orthopedic walking boots, back support and/or braces, foot and/or ankle supports, knee braces, or elbow braces, among others.

The cervical collar 10 is configured to support the head in a stable, stationary position. It may be used in cases where the user has suffered injury to or undergone surgery for the cervical spine. The cervical collar 10 may comprise an anterior component 2 and a posterior component 4 that surround the user's neck and/or head. The anterior and posterior components 2, 4 may comprise a rigid and/or a semi-rigid plastic that may provide structural support to the user's head and neck. The anterior and/or posterior components 2, 4 may be adjustable (e.g., adjustable height and which the chin and jaw are elevated) to improve the fit and comfort of the user. A lining 6 may be disposed between the anterior and posterior components 2, 4 to increase the comfort of the user while wearing or using the device. The anterior and posterior components 2, 4 may be separated by a gap 8 having a width through which the user's head and/or neck may pass to facilitate securing the cervical collar 10 to the user. The width of the gap 8 may then be decreased or closed to position the user's head in the desired position upon the collar 10.

FIG. 2 illustrates a portion of a hook strap 12 that may be used as part of the hook and loop fastener 5 to secure two structures, such as the anterior component 2 and the posterior component 4, to one another. The hook strap 12 of the hook and loop fastener 5 may include a base 32 comprising a band or segment having a plurality of fastening elements 42 comprising injection molded hooks 14 integrally formed with and extending from a first side 34 of the base 32, the second side 36 opposite the first side 34 being adhered or otherwise attached to a structure of the collar 10, such as the anterior component 2. The loop strap of the hook and loop fastener 5 may also comprise a band or segment that includes micro loop structures configured to interlock with the injection molded hooks 14 of the hook strap 12. The loop strap may be configured to attach to the hook strap 12 to secure the structure (e.g., the anterior component 2 and the posterior component 4) together. The loop strap may extend from the hook strap 12 through a strap loop in the posterior component 4 or from the posterior component 4 to connect with the hook strap 12. Portions of the hook and loop fastener 5, such as the hook strap 12, have commonly been attached to structures by applying an adhesive between the second side 36 and the surface of the structure.

Many orthopedic devices use rigid plastics or metals, preventing hook and loop fasteners 5 from adequately attaching to the device via a sewing method. Orthopedic devices, such as the cervical collar 10 illustrated, are also benefitted by the omission of any fasteners comprising ferromagnetic metals (e.g., iron, cobalt, manganese, and nickel), such as screws, pins, and staples, used to secure the hook and loop fasteners 5, to enable the orthopedic device to be worn during medical imaging procedures, such as magnetic resonance imaging (MRI), x-ray imaging, and computed tomography (CT) scans. Many hook and loop fasteners 5 are secured to orthopedic devices via adhesive, but may delaminate over many fastening and unfastening cycles or under hot and/or humid conditions. Nauseous odors arising from some adhesives may be particularly offensive to users of cervical collars 10 because the adhesive is located in a position especially close to the user's face and nose. Thus, there is a need for hook and loop fasteners that may be attached to orthopedic devices without the use of a sewn connection, a ferromagnetic fastener connection, or an adhesive connection.

### Fixation Member

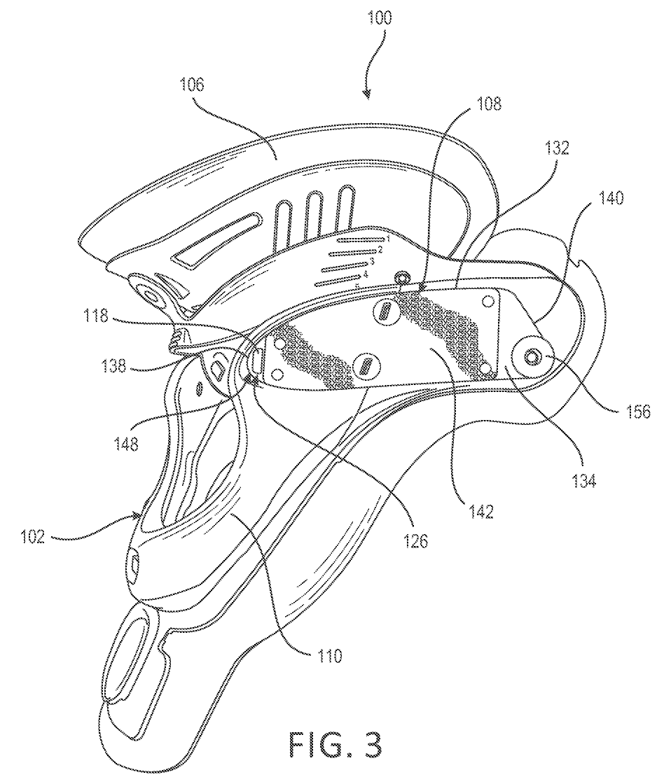

FIG. 3 illustrates an orthopedic device 100 comprising a cervical collar. The orthopedic device 100 may include a fixation member 108 comprising a plurality of fastening elements 142 having injection molded hooks similar to hooks 14 of FIG. 2. The fixation member 108 may be attached to the orthopedic device 100 without the use of a sewn connection, a ferromagnetic fastener connection, or an adhesive connection. Rather, the plurality of fastening elements 142 may only be attached to the orthopedic device 100 through mechanical connections and/or welded attachments. The fixation member 108 and orthopedic device 100 may comprise similar characteristics and features as the fastener member and orthopedic devices recited in U.S. Pat. No. 9,474,334, granted on October 25, 2016, U.S. Pat. No. 10,245,170, granted on April 2, 2019, and U.S. Pat. No. 10, 945872, granted on March 16, 2021 (whereby FIG. 1 is shown), which are herein incorporated by reference. Although the illustrated orthopedic device 100 comprises a cervical collar, one skilled in the art will understand that the fixation member 108 may be employed in other orthopedic devices 100, including orthopedic walking boots, back supports and/or braces, foot and/or ankle supports, knee brace, elbow braces, or other orthopedic device or suitable article 100.

The orthopedic device 100 may comprise an anterior component 102, a posterior component (not shown, but which may be similar to posterior component 2 illustrated in FIG. 1) configured to be secured to the anterior component 102, and a fixation member 108 attached to the anterior 102 or posterior components and configured to facilitate securement of the anterior component 102 to the posterior component. The orthopedic device 100 may also comprise a lining 106 between the anterior 102 and posterior components configured to increase the user's comfort. The anterior component 102 may comprise multiple structures, including a first part 110 and a second part 112. The first part 110 may be connected directly to the second part 112.

The second part 112 may be disposed towards an inner surface of the device relative to the first part 110, such that the second part 112 is disposed posteriorly of the first part 110 between the user and the first part 110 when the user wears the orthopedic device. The first and second parts 110, 112 may comprise a plastic material. The second part 112 may comprise a less rigid material compared to the first part 110, such that the first part may provide greater structural support. In contrast, the second part 110 may provide a softer material to interface with the user and prevent discomfort. For example, the first part 110 may comprise ethylene vinyl acetate (EVA), and the second part 112 may comprise nylon.

The fixation member 108 may be connected to the anterior component 102 of the orthopedic device 100 at the first part 110, the second part 112, and/or any other structure of the anterior component 102. Although the following description primarily describes the fixation member 108 as attaching to the anterior component 102 of the orthopedic device 100, one skilled in the art will appreciate that the fixation member 108 may alternatively be attached to the posterior component or other component of the orthopedic device 100. Any reference to connections or features existing on or between the fixation member 108 and the anterior component 102 may similarly be present on or between the fixation member 108 and the posterior component or other component of the orthopedic device 100 or other article.

Specifically, the fixation member 108 may be attached to any component of the orthopedic device 100 configured to be secured to another component of the orthopedic device 100. Similarly, the fixation member 108 may be attached to other structures that do not form part of an orthopedic device, such that the fixation member 108 may be attached to a first structure configured to be secured relative to a second structure. The fixation member 108 may comprise any material (e.g., plastic) that is suitable for forming injection molded parts. For example, the fixation member 108 may comprise a thermoplastic or thermoset material. The fixation member 108 may comprise the same material as the first and/or second parts 110, 112, or may comprise a material that is more rigid or less rigid than the first and/or second parts 110, 112.

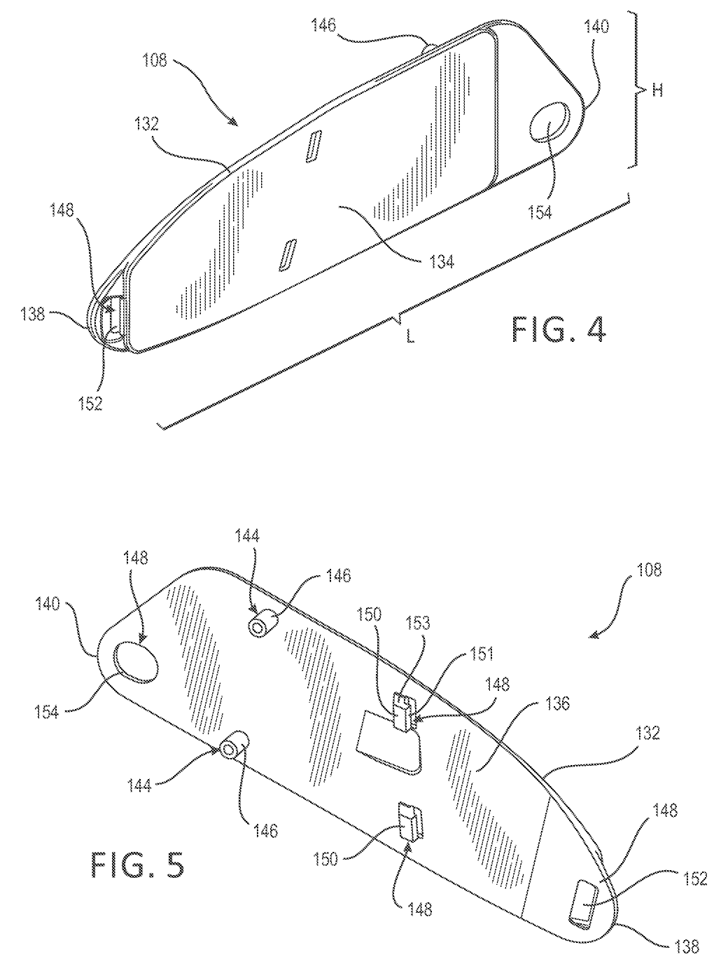

FIGS. 4 and 5 more clearly illustrate the fixation member 108 of the orthopedic device 100. The fixation member 108 may include a base 132 having first and second sides 134, 136, and a first end 138 disposed opposite a second end 140. As illustrated, the fixation member 108 may comprise a generally oblong shape (i.e., one side having a longer length and another) with a length L greater than a height H. According to one embodiment, the fixation member 108 may comprise a length L to height H ratio of approximately 1.5:1, approximately 2:1, approximately 2.5:1, approximately 3:1, approximately 4:1, or approximately 5:1, or may comprise a length L to height H ratio within a range having any two of the foregoing as endpoints. The fixation member 108 may comprise a profile delimiting the perimeter of the first and/or second sides 134, 136 having one of various shapes, such as a generally square, rectangular, triangular, polygonal, circular, elliptical, or oval shape, or may have an irregular shape.

The profile, or portions thereof, delimiting the perimeter of the second side 136 may approximate or conform to a recess (not shown) formed in the anterior component 102 to which the fixation member 108 may be attached, such that the fixation member 108 fits within or conforms to the surface of the recess. Portions of the anterior component 102 adjacent to and extending beyond the recess may thereby be used to guide the fixation member 108 to cooperate with and attach to the anterior component 102. The recess may span across both the first and second parts 110, 112 of the anterior component 102. The fixation member 108 may similarly span across the first and second parts 110, 112. In embodiments wherein the fixation member 108 is attached to both the first and second parts 110, 112, the fixation member 108 may beneficially facilitate connection of the first and second parts 110, 112 to each other, preventing the first and second parts 110, 112 from separating.

The fixation member 108 may have a relatively small thickness extending between the first and second sides 134, 136, and may have a thickness within a range from approximately 0.5 mm to approximately 5 mm, or from approximately 1 mm to approximately 4 mm, or from approximately 2 mm to approximately 3 mm, or may have a thickness having any two of the foregoing as endpoints.

A plurality of fastening elements 142 (which may include but are not limited to micro hooks 14 illustrated in FIG. 2) may extend from a first side 134 of the base 132 and may be integrally formed with the base 132, such that the plurality of fastening elements 142 is formed from the same material as the base 132. The plurality of fastening elements 142 may comprise injection molded hooks similar to injection molded hooks 14 configured for use in a hook and loop fastening system, or may comprise any other integrally formed quick adjustment mechanism configured to secure a first structure to a second structure.

Fastening elements 142 that are integrally formed with the base 132 are particularly beneficial in that they are less likely to detach from device 100 as long as the fixation member 108 is attached to the device 100. The plurality of fastening elements 142 may extend over all of the first side 134, or over only a portion of the first side 134, including a portion covering the majority of the first side 134 or a portion covering a minority of the first side 134. The plurality of fastening elements 142 may be disposed over a single continuous portion or may extend over multiple separate and distinct portions of the first side 134, such as over two, three, four, five, or more than five separate and distinct portions of the first side 134. The first side 134 may comprise two or more portions with a plurality of fastening elements 142 (e.g., injection molded hooks) separated by a portion not having a plurality of fastening elements 142. Portions of the first side 134 not having fastening elements 142 may have a smooth surface or may have a non-smooth surface, such as a rough surface, or may have a surface shaped to conform to, interface with, or cooperate with the underlying structure.

### Welded Attachments and Mechanical Connections

The second side 136 of the base 132 may comprise one or more features configured to secure the fixation member 108 to the anterior component 102. As used herein, whenever the fixation member 108 is described as being attached or connected to a structure of the anterior component 102, such as the first part 110, one skilled in the art will understand that the fixation member 108 may be attached or connected similarly to another structure, such as the second part 112 or other structure of the anterior component 102.

The fixation member 108 may comprise one or more elements configured for mechanically connecting and/or welding the fixation member 108 to the anterior component 102. By combining an integrally formed fixation member 108 with a fastener comprising injection molded hooks with mechanical connections and/or welded attachments, a fastening system comprising a hook and loop fastener may be incorporated into an orthopedic device 100 without adhesive. That is, the connection between the fixation member 108 and the anterior component 102 may beneficially be completely free of adhesive. Indeed, according to some embodiments, the orthopedic device 100 may be entirely free of adhesive. In this manner, the orthopedic device 100 and the fastening system comprising the hook and loop fastener may avoid problems associated with adhesives, including delamination of the hook and loop fastener from the device 100 or destruction of the hook and loop fastener (e.g., by tearing a nylon strap used therein). A fastening system using mechanical connections and welded attachments rather than adhesive may also provide a more durable connection. Reducing the number of individual components employed by the device 100 may simplify the assembly process.

The fixation member 108 may comprise one or more first fixation elements 144 extending from the second side 136 of the base 132. The first fixation elements 144 may be arranged to form a welded attachment with corresponding attachment features 126 formed on the anterior component 102 structure. The first fixation elements 144 may be integrally formed with the base 132 of the fixation member 108 (e.g., through an injection molded process). According to an embodiment, as the fixation member 108 is assembled with the anterior component 102, the first fixation elements 144 of the fixation member 108 may be welded to the attachment features 126 formed on the anterior component 102.

The first fixation elements 144 may be welded to attachment features 126 through various welding processes, including heat staking, ultrasonic welding, laser welding, spin or friction welding, solvent welding, extrusion welding, hot plate welding, or other welding processes. As illustrated in FIG. 5, the first fixation elements 144 may comprise heat stakes 146 configured to affix to the attachment features 126 of the anterior component 102 by a heat staking process. As illustrated, the fixation member 108 may comprise two or more first fixation elements 144.

FIG. 6 illustrates the attachment features 126 that may be formed in the second part 112. The attachment features 126 may be configured to cooperate with the first fixation elements 144 to form a weld. According to an embodiment, the attachment features 126 may be configured to receive the first fixation elements 144. For example, the attachment features 126 may form openings 128 through which the heat stakes 146 may protrude. After the heat stakes 146 have been inserted through the openings 128, the fixation member 108 may be affixed to the second part 112 by applying a heat staking process to the heat stakes 146 (described more fully below).

According to an embodiment, the attachment features 126 may comprise heat stakes integrally formed with the second part 112 and having a greater width than the heat stakes 146 of the first fixation elements 144, the heat stakes of the attachment features 126 extending from an inner surface 124 of the second part 112 and including a lumen through which the heat stakes 146 may be inserted. After the heat stakes 146 of the first fixation elements 144 are inserted through the lumen of the heat stakes of the attachment features 126, both the heat stakes 146 and the heat stakes of the attachment features 126 may be welded together through a heat staking process.

The first fixation elements 144 may extend from only a portion of the base 132, such as a portion of the base 132 lying near or adjacent to the second end 140. The first fixation elements 144 may be attached to only one structure of the anterior component 102, such as the first or second parts 110, 112. In such embodiments, the first fixation elements 144 may be formed on a portion of the second side 136 closer to the second end 140 of the base 132 than the first end 138 of the base 132. Alternatively, the first fixation elements 144 may be used to form a welded attachment with multiple structures of the anterior component 102, such as the first and second parts 110, 112 of the anterior component 102.

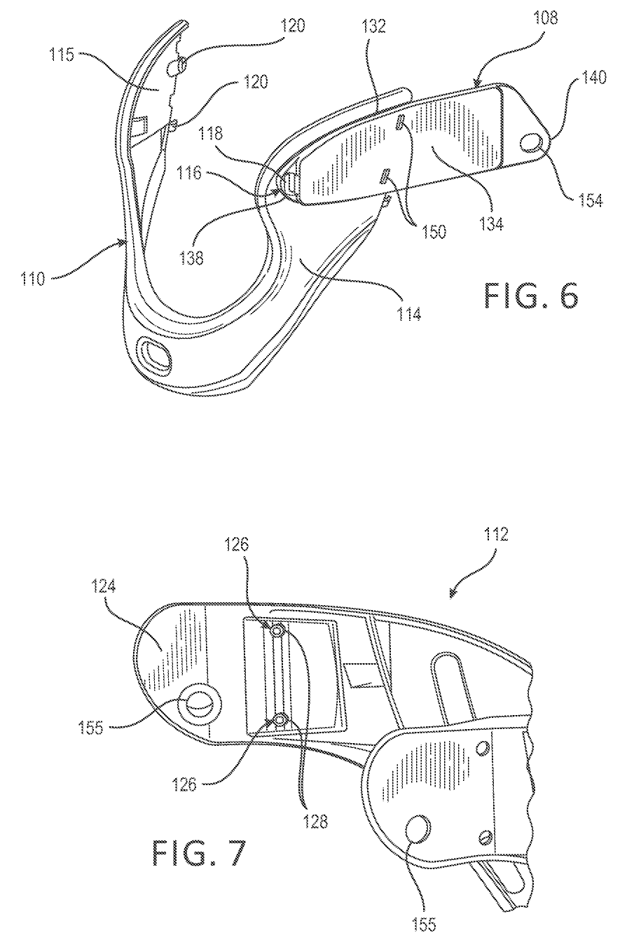

FIG. 7 illustrates the fixation member 108 mechanically connected to the first part 110. The base 132 may comprise one or more second fixation elements 148 extending therefrom. The second fixation elements 148 may extend from the second side 136 of the base 132 or may extend at or near the first and/or second ends 138, 140 of the base 132. The second fixation elements 148 may be integrally formed with the base 132 of the fixation member 108 and may be formed through cutting or otherwise deforming the base 132 of the fixation member 108. The second fixation elements 148 may be arranged to form a mechanical connection with a corresponding connection feature 116 of the anterior component 102. The mechanical connection may comprise a releasable connection, such that in at least some embodiments, the fixation member 108 may be detached from a structure of the anterior component 102 to which the fixation member 108 is only mechanically connected.

The second fixation elements 148 may be configured to interlock with a corresponding connection feature 116 of the anterior component 102. The second fixation elements 148 may comprise one or more snap hooks 150. The snap hooks 150 may be configured to interlock with a corresponding connection feature 116 comprising a connection opening 120. The snap hook 150 is inserted through the connection opening 120 until a distal edge 151 of the snap hook 150 protrudes from an inner surface 115 of the first part 110 to form the mechanical connection. The distal edge 151 of the snap hook 150 may extend over the inner surface 115 of the first part 110 to prevent the snap hook 150 from exiting the connection opening 120.

According to an embodiment, the snap hook 150 may be released from the opening 120 by deforming the stem 153 of the snap hook 150 to move the distal edge 151 out from over the inner surface 115 and allow the snap hook 150, including the distal edge 151, to pass back through the connection opening 120.

The second fixation elements 148 may comprise a slot 152. The slot 152 may comprise an opening formed at or near the first and/or second ends 138, 140 of the base 132 or on another portion of the base 132. The slot 152 may be configured to receive a connection feature 116 comprising a protuberance 118. The protuberance 118 may extend from a structure of the anterior component 102, such as the outer surface 114 of the first or second parts 110, 112.

The protuberance 118 may protrude from a first structure of the anterior component 102 and may extend in a direction generally away from a second structure of the anterior component 102 to which the fixation member 108 is configured to attach. For example, the protuberance 118 may protrude from the outer surface 114 of the first part 110 in a direction generally away from the second part 112. In this manner, when the fixation member 108 is attached to the second part 112, the slot 152 and protuberance 118 may cooperate to pull the first part 110 towards the second part 112 and beneficially bias the first and second parts 110, 112 towards one another to facilitate and maintain the connection therebetween.

According to an embodiment, the protuberance 118 may comprise a snap hook that may interlock with the slot 152 to secure the fixation member 108 to the first part 110. The snap hook of the protuberance 118 may comprise a releasable attachment of the fixation member 108 to the first part 110.

The second fixation elements 148 may be configured to receive a connection element connecting the fixation member 108 to the anterior component 102. According to an embodiment, the second fixation elements 148 may include a first aperture 154 comprising an opening formed in the first and/or second ends 138, 140 of the base 132 or another portion of the base 132. The first aperture 154 may be configured to cooperate with a connection feature 116 that may include a second aperture 155 comprising an opening formed in the anterior component 102, such as the second part 112. The first and second apertures 154, 155 may be configured to receive a pin 156 that mechanically connects the fixation member 108 to the anterior component 102 of the orthopedic device 100. The pin 156 may comprise a non-ferromagnetic material, such as a rigid or semi-rigid plastic material.

The above welded attachments and mechanical connections may facilitate the connection between the fixation member 108 and the anterior component 102. The various first and second fixation elements 144, 148 may be disposed at or near the first and second ends 138, 140 of the base 132 or over various portions of the second side 136 of the base 132 to provide the welded attachment and mechanical connections between the fixation member and any or all of the structures of the anterior component 102, as desired.

For example, the fixation member 108 may connect to a first structure of the anterior component 102 (e.g., the first or second parts 110, 112) only through one or more mechanical connections and may connect to a second structure of the anterior component 102 (e.g., the other structure of the first or second parts 110, 112) by a welded attachment and/or a mechanical connection. In another example, the fixation member 108 may connect to a first structure of the anterior component 102 (e.g., the first or second parts 110, 112) only through one or more welded attachments and may connect to a second structure of the anterior component 102 (e.g., the other structure of the first or second parts 110, 112) by a welded attachment and/or a mechanical connection.

The fixation member 108 may be connected to multiple structures of the anterior component 102 (e.g., the first and second parts 110, 112) only through welded attachments, or only through mechanical connections, or may be connected to each of the multiple structures through both welded attachments and mechanical connections. According to an embodiment, the fixation member 108 may be attached to one of the structures of the anterior component 102 (e.g., the first or second parts 110, 112), but not to any other structure of the anterior component 102.

### Heat Staking Process

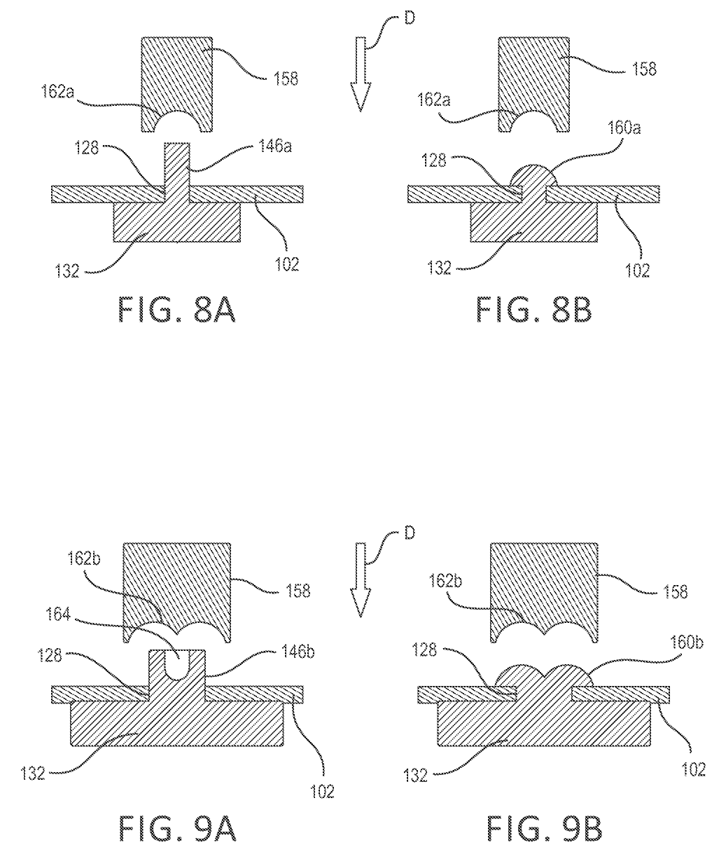

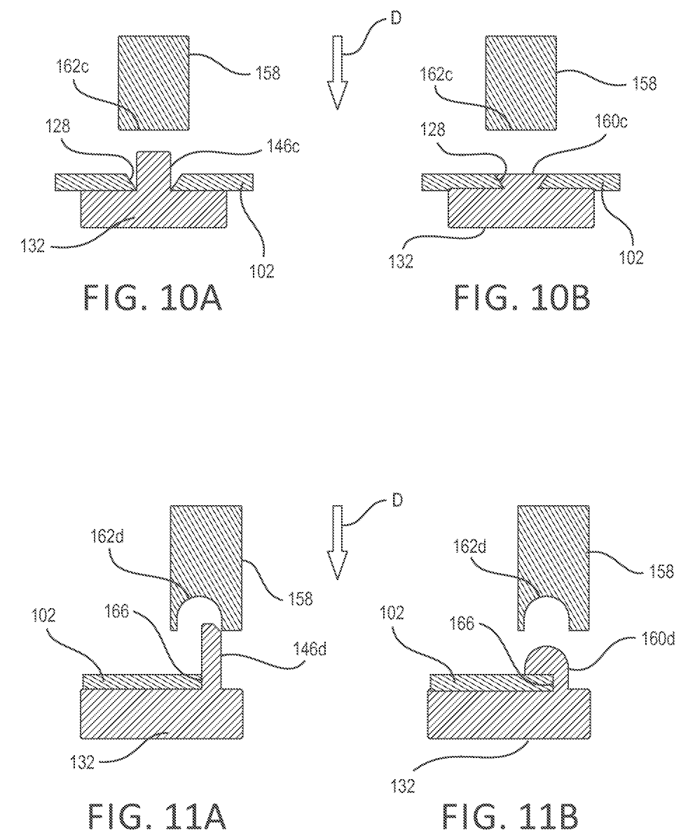

FIGS. 8A-8B, 9A-9B, 10A-10B, 11A-11B further illustrate how the heat stakes 146 illustrated in FIG. 5 may, through exertion of heat, form a welded attachment to the anterior component 102. The heat stakes 146a, 146b, 146c, 146d may be similar to the heat stakes 146 described above. The heat stakes 146a, 146b, 146c, 146d may extend from the base 132 of the fixation member 108 through the openings 128 formed in the anterior component 102. A forming tool 158 comprising a thermal tip 162a, 162b, 162c, 162d may then be disposed over the heat stakes 146a, 146b, 146c, 146d (see FIGS. 8A, 9A, 10A, and 11A), the thermal tip 162a, 162b, 162c, 162d being heated to a sufficient temperature (e.g., a temperature at or above the glass transition temperature of the heat stakes 146a, 146b, 146c, 146d) to melt the heat stakes 146a, 146b, 146c, 146d. The forming tool 158 may then descend in direction D to create localized heating and melt the heat stakes 146a, 146b, 146c, 146d to form a pin 160a, 160b, 160c, 160d that conforms to the shape of the thermal tip162a, 162b, 162c, 162d. The thermal tip 162a, 162b, 162c, 162d is then cooled (e.g., to a temperature below the glass transition temperature of the heat stakes 146a, 146b, 146c, 146d) to enable the pin 160a, 160b, 160c, 160d to solidify.

Because the pin 160a, 160b, 160c, 160d has a width greater than the opening 128, the fixation member 108 is thereby welded to, forming a permanent connection with the anterior component 102. The forming tool 158 is then raised in a direction opposite direction D to reveal the pin 160a, 160b, 160c, 160d (see FIGS. 8B, 9B, 10B, and FIG. 11B).

Generally, the temperature of the thermal tip 162a, 162b, 162c, 162d may not be sufficient to melt a portion of the anterior component, such that the pin 160a, 160b, 160c, 160d is formed only from the material of the heat stakes 146a, 146b, 146c, 146d. Alternatively, the thermal tip 162a, 162b, 162c, 162d may attain a sufficient temperature to melt a portion of the anterior component 102 surrounding the heat stakes 146a, 146b, 146c, 146d and allowing the material of the melted heat stakes 146a, 146b, 146c, 146d and the surrounding portions of the anterior component 102 to mix and fuse when forming the pin 160a, 160b, 160c, 160d of the weld.

The thermal tip 162a, 162b, 162c, 162d may comprise various configurations and shapes to provide the desired size and shape of the pin 160a, 160b, 160c, 160d. The shape of the pin 160a, 160b, 160c, 160d may comprise a semi-spherical shape, such as that shown in FIGS. 8A-8B and 11A-11B. According to an embodiment, the heat stakes 146b may be relatively thick and may comprise a hollow lumen 164 to form a rim-shaped pin 160b. The rim-shaped pin 160b may enable the pin to extend over a larger portion of the anterior component 102 compared to other pins 160a, 160c, 160d, and may provide a more secure connection.

According to another embodiment, the heat stake 146c may be formed into a flush pin 160c that has an upper surface that is co-planar with a surface of the anterior component 102. The flush pin may beneficially create a smooth inner surface along the welded attachment between the anterior component 102 and the fixation member 108. Finally, the welded attachment may comprise a roll-over pin 160d formed from a heat stake 146d that, rather than being inserted through the opening 128 of the anterior component 102 may extend around the edge 166 of the anterior component 102 to secure the fixation member 108 thereto.

It is understood that not all objects or advantages might be achieved in every embodiment of the disclosure. Those skilled in the art will recognize that the fixation member and/or orthopedic device can be embodied or implemented to achieve or optimize one particular advantage or group of advantages shown here, without necessarily achieving other objects or benefits suggested or described.

The skilled person will see the interchangeability of various features disclosed. Besides the options described here, other known equivalents for each feature can be combined by one of ordinary skill in this field to develop and operate the fixation members and orthopedic devices according to the principles of this disclosure.

The skilled person will also understand that these features can be adapted to other methods and types of orthopedic devices or applications. It is intended that this disclosure not be limited by the specific embodiments described above and may be extended to other applications that employ the features discussed.

CLAIMS

1. A fixation member configured for affixation to a structure, the fixation member comprising:
a base including first and second sides, and first and second ends;
a plurality of fastening elements integrally formed with and extending from a first side of the base;
a first fixation element extending from the second side of the base and arranged to form a welded attachment with an attachment feature of the structure; and a second fixation element extending from the base and arranged to form a mechanical connection with a connection feature of a structure;
wherein the fixation member is formed from plastic.

2. The fixation member of claim 1, wherein the first fixation element is securable to the attachment feature via a heat staking, ultrasonic welding, laser welding, spin or friction welding, solvent welding, extrusion welding, or hot plate welding process.

3. The fixation member of claim 1, wherein the first fixation element is arranged in a first shape and is configured, upon exertion of heat, to form a second shape to interlock with the attachment feature of the structure.

4. The fixation member of claim 1, wherein the first fixation element comprises a heat stake.

5. The fixation member of claim 1, wherein the first fixation element is disposed closer to the second end of the base than the first end of the base.

6. The fixation member of claim 1, wherein the fixation member comprises two or more first fixation elements.

7. The fixation member of claim 1, wherein the second fixation element comprises a snap hook extending from the second side of the base, the second fixation element configured to mechanically affix to the connection feature, and wherein the connection feature forms a connection opening.

8. The fixation member of claim 1, wherein the second fixation element defines a slot extending from the base at a first end, wherein the slot is configured to receive the connection feature comprising a protuberance of the structure.

9. The fixation member of claim 1, wherein the plurality of fastening elements protrudes from a surface extending over a majority of the first side.

10. The fixation member of claim 1, wherein the plurality of fastening elements protrudes from a surface extending over a minority of the first side.

11. The fixation member of claim 1, wherein the base defines an aperture formed at or near the second end arranged to receive a pin.

12. The fixation member of claim 1, wherein the fixation member comprises a thermoplastic or a thermoset material.

13. An orthopedic device comprising:
an anterior component; a posterior component configured to be secured to the anterior component; and
a fixation member attached to the anterior or posterior components and configured to facilitate securement of the anterior component to the posterior component, the fixation member including:
a base including first and second sides and first and second ends;
a plurality of fastening elements integrally formed with and extending from a first side of the base;
a first fixation element extending from the second side of the base and arranged to form a welded attachment with an attachment feature of a structure; and a second fixation element extending from the base and arranged to form a mechanical connection with a connection feature of the structure;
wherein the fixation member comprises a plastic.

14. The orthopedic device of claim 13, wherein the first fixation element is welded to the attachment feature via a heat staking, ultrasonic welding, laser welding, spin or friction welding, solvent welding, extrusion welding, or hot plate welding process.

15. The orthopedic device of claim 13, wherein the first fixation element is arranged in a first shape and is configured, upon exertion of heat, to form a second shape to interlock with the attachment feature of the structure.

16. The orthopedic device of claim 13, wherein the anterior component comprises a first part and a second part.

17. The orthopedic device of claim 16, wherein the first part is connected to the second part.

18. The orthopedic device of claim 16, wherein the fixation member is connected to both of the first and second parts.

19. The orthopedic device of claim 18, wherein the fixation member is welded to one of the first and second parts and forms a mechanical connection with another part of the first and second parts.

20. A method for attaching a fixation member to a structure, comprising the steps of:
providing an attachment feature and a connection opening on the structure; attaching the fixation member to the structure, the fixation member including a base having first and second sides, a plurality of fastening elements extending from a first side of the base, and first and second affixation elements extending from the base; welding the fixation member to the structure by affixing the first fixation element to the structure about the attachment feature; and
mechanically attaching the fixation member to the structure by affixing the second affixation element to the structure about the connection feature.
